(19) United States
(12) Patent Application Publication
(10) Pub. No.: US 2010/0240549 A1
(43) Pub. Date: Sep. 23, 2010

(54) SPECIFIC AMPLIFICATION OF TUMOR SPECIFIC DNA SEQUENCES

(75) Inventor: Stephen A. Brown, South Burlington, VT (US)

Correspondence Address: KENYON & KENYON LLP, ONE BROADWAY, NEW YORK, NY, 10004, US

(73) Assignee: THE TRUSTEES OF COLLUMBIA UNIVERSITY IN THE CITY O, New York, NY (US)

(21) Appl. No.: 12/666,167

(22) PCT Filed: Jun. 22, 2008
(86) PCT No.: PCT/US08/67822

§ 371 Date: May 10, 2010

Related U.S. Application Data
(60) Provisional application No. 60/936,789, filed on Jun. 22, 2007.

(51) Int. Cl. C40B 30/04; C12P 19/34; C12Q 1/68; C40B 50/00; C40B 40/06

(52) U.S. Cl. 506/9; 435/91.2; 435/6; 506/23; 506/16

(57) ABSTRACT

The present invention provides methods for cancer detection and diagnosis. The present invention provides a method of selectively amplifying hypomethylated tumor DNA sequences derived from a subject for detection of cancer. This method utilizes differential methylation to allow for the selective amplification of tumor specific sequences from DNA mixtures that contain a high proportion of normal host DNA. The invention also provides methods of using the amplified tumor DNA sequences for evaluation of methylation.

### FIELD OF THE INVENTION

The present invention encompasses methods for cancer detection and diagnosis.

### BACKGROUND OF THE INVENTION

Existing methods for cancer screening are costly and largely ineffective, and as a consequence, most cancers are detected at a late and poorly treatable stage. This is particularly true for ovarian cancer. Therefore, the need for new methods for cancer screening is widely recognized. A large number of recent publications have documented the existence of circulating nucleic acids (CNA) in the body fluids of patients with cancer, and various strategies for using CNA for detecting, following and prognosticating cancer have been considered (reviewed in (Fleischhacker and Schmidt 2007)). The simplest approach has been to compare the total amount of CNA between cancer patients and controls. Such studies have generally found that cancer patients have more CNA than cancer-free controls, but it has been impossible to show a good correlation between tumor size, stage location or type and total CNA concentration. Simple quantitation is further complicated by the fact that many other conditions such as chronic inflammation and chronic obstructive pulmonary disease (COPD) are associated with increased levels of CNA.

A more promising approach to the use of CNA has been the detection of cancer-specific sequence changes. Acquired mutations in K-RAS and/or P53 have been identified in CNA of patients with pancreatic, colorectal, lung and ovarian cancers (Fleischhacker and Schmidt 2007). Several authors have considered the possibility of using the detection of known cancer mutations as a method for cancer screening. In one such study, the authors found that screening for K-RAS mutations in CNA of patients who underwent colonoscopy was useful in predicting who would have colonic malignancy (Kopreski, Benko et al. 2000). Other studies have provided conflicting results, making it clear that no single mutation will provide robust cancer detection (Yakubovskaya, Spiegelman et al. 1995; Trombino, Neri et al. 2005).

Another avenue that has been considered is the analysis of microsatellite instability, which provides an avenue for finding cancer related sequence changes without targeting specific, known mutations. Several studies have shown that microsatellite changes are present in circulating DNA even at early stages of breast and lung cancer (Chen, Bonnefoi et al. 1999; Sozzi, Musso et al. 1999; Sozzi, Conte et al. 2001).

Epigenetic changes in DNA sequence offer a third avenue for specific amplification of cancer DNA from CNA specimens. Thus far, more than 40 publications have reported efforts to detect cancer-related alterations in methylation in CNA of blood and body fluids of a wide variety of cancer patients (Fleischhacker and Schmidt 2007). In almost all such studies, one or several CpG islands that are frequently hypermethylated in cancer were queried through the use of methylation-specific PCR (Herman, Graff et al. 1996), and most studies reported some degree of success. Depending on which CpGs were analyzed, it was almost always possible to detect cancer related changes in some proportion of subjects with a given type of cancer. In general, one can conclude from this work that, while altered methylation of specific loci is frequently present in CNA of cancer patients, no particular locus promises to be the basis of a robust test. In a review of cancer epigenetics, it is suggested that large-scale analysis of methylation in CNA would solve the problem of examining only one or several loci, and concludes that microarray based methods of methylation analysis hold great promise for cancer detection (Laird 2005). In order to achieve large-scale detection of cancer related methylation changes in CNA, methods for methylation specific DNA amplification as well as microarray technology for the detection of methylation differences are necessary.

Because tumor DNA can be routinely recovered from cell-free plasma of subjects with a variety of different types of cancer (including ovarian), it provides an attractive means for assessing the presence of malignancy. However, the use of circulating DNA for cancer detection has been hampered by two major problems. First, circulating DNA (or “CNA”) is always contaminated by substantial amounts of normal host DNA. Therefore, methods to specifically amplify tumor DNA generally rely on prior knowledge of genomic differences between tumor and normal, such as cancer specific mutations or alterations of methylation. This constraint severely limits the number of loci that can be amplified. Second, tumors are highly diverse, so that the detection of only one or several tumor specific genomic alterations is unlikely to provide a robust method for cancer detection. The present invention provides a solution to these two problems by allowing for the general but highly selective differential amplification of hypomethylated tumor DNA when it is mixed with normal host DNA and simultaneous evaluation of methylation of a large number (>10⁵) of loci. Thus, the present invention provides a novel approach to cancer screening by high-throughput analysis of methylation of circulating DNA.

### SUMMARY OF THE INVENTION

The present invention relates to methods for the diagnostic evaluation and prognosis of cancer, especially ovarian cancer. The present invention provides a method for selective amplification of hypomethylated DNA from the serum or plasma of a subject comprising: digesting the DNA with a methylation sensitive enzyme; ligating the digested DNA with a linker; subjecting the digested DNA to linker-mediated PCR amplification to obtain PCR products; purifying the PCR products; and amplifying the purified PCR products. In one embodiment, the amplification of the purified PCR products is accomplished by circularizing the amplified PCR products; and subjecting the closed circular molecules to isothermal rolling circle amplification to selectively amplify hypomethylated DNA to produce methylation-sensitive representations from a DNA sample.

In one embodiment the invention provides a method for selective amplification of hypomethylated DNA from the serum or plasma of a subject comprising: digesting the DNA with a methylation sensitive enzyme; ligating the digested DNA with a linker; subjecting the digested DNA to linker-mediated PCR amplification to obtain PCR products; removing linker and primer DNA from the amplification products; circularizing the amplified PCR products; digesting the DNA with a second restriction enzyme that digest the DNA at the site where the linker has been added; removing linkers from the digested DNA; self ligating the digested DNA to form closed circular molecules; subjecting the circularized molecules to exonuclease digestion to reduce any uncircularized DNA to single nucleotides; and subjecting the closed circular molecules to isothermal rolling circle amplification to selectively amplify hypomethylated DNA to produce methylation-sensitive representations from a DNA sample.

DNA prepared by the above method may then be hybridized to a custom made oligonucleotide microarray. In one embodiment, the oligonucleotides on the array corresponds to one of the DNA restriction fragments or portions thereof that could be theoretically created during the first digestion step using the methylation sensitive enzyme. The intensity of signal at each array address is dependent on the amount of probe (labeled DNA) that corresponds to the address. Thus, array addresses for which signal intensity is high are relatively less methylated. Through a comparison of microarray data from normal controls to those with cancer, a typical methylation profile of cancer is derived. Methylation/microarray results from samples obtained from subjects where cancer status is unknown is compared with the body of normal data. Deviations from normal are indicative of cancer.

In a specific embodiment of the invention, a method is provided for the selective amplification of tumor DNA derived from a subject sample. The method of the invention comprises (i) digesting the DNA isolated from a subject sample with a methylation specific enzyme; (ii) ligating linkers to the ends of the digested DNA; (iii) subjecting the digested DNA to linker-mediated PCR amplification; (iv) purifying the PCR products, (v) digesting the purified PCR products with a restriction enzyme that recognizes a restriction site contained partly or entirely within the linkers; (vi) circularizing the purified PCR products; and (vii) subjecting the products from step (vi) to isothermal rolling circle amplification to selectively amplify tumor DNA to produce methylation-sensitive representations from tumor DNA. In a further embodiment, the PCR primers used in the linker-mediated PCR are conjugated to a moiety useful in the subsequent purification of the PCR products. In one embodiment the PCR primers are conjugated to biotin. In a further embodiment, the PCR products are purified by binding the moiety to a support. In one embodiment the linker-mediated PCR primer is biotinylated and the resulting PCR products are purified using a biotin binding protein (e.g., avidin or streptavidin) linked to a support (e.g., agarose, sepharose, or magnetic beads). In one embodiment the PCR products are freed from the support by cleaving with a restriction enzyme that recognizes a restriction site created by the ligation of the linker to the DNA digested with the methylation sensitive enzyme. In one embodiment the linker is cleaved with MluI.

In another specific embodiment of the invention, a method is provided for the selective amplification of tumor DNA derived from a subject sample. The method of the invention comprises (i) digesting the DNA isolated from a subject sample with a methylation specific enzyme; (ii) ligating linkers to the ends of the digested DNA; (iii) subjecting the digested DNA to linker-mediated PCR amplification to obtain amplified PCR products; (iv) digesting the amplified PCR products with a restriction enzyme that cleaves the DNA at the site the linkers were added; (v) removing the cleaved linkers from the PCR products; (vi) circularizing the PCR products; (vii) subjecting the circularized PCR products to exonuclease digestion to digest remaining linear DNA molecules; and (viii) subjecting the products from step (vii) to isothermal rolling circle amplification to selectively amplify tumor DNA to produce methylation-sensitive representations from tumor DNA.

The present invention further provides a method for identifying tumor-specific hypomethylated DNA regions comprising, (i) separately preparing methylation-sensitive representations from tumor and normal DNA using a method described above; (ii) labeling the tumor DNA and control DNA to produce labeled tumor DNA probes and labeled normal DNA probes; (iii) hybridizing the labeled DNA probes to arrays of oligonucleotides, wherein said array of oligonucleotides corresponds to predicted restriction fragments, or portions thereof, for a given methylation-sensitive enzyme; (iv) comparing the relative intensity of the normal and tumor derived probes with each other to identify oligonucleotides that detects the differential amount of tumor DNA probe; (v) identifying the hybridized oligonucleotide from step (iv) as a corresponding to tumor-specific hypomethylated region. In one embodiment, the two representations are labeled with different labels (e.g., different fluorochromes) and hybridized to the same array. In another embodiment, the labeled probes are hybridized to separate microarrays.

The present invention further provides a method for detecting cancer in a subject. The method comprises preparing methylation-sensitive representations from a patient derived sample using a method described above followed by labeling the DNA to produce labeled tumor DNA probes. The labeled DNA probes are hybridized to an oligonucleotide array, wherein said array of oligonucleotides correspond to predicted restriction fragments, or portions thereof, for the methylation specific enzyme. Such hybridization will lead to the generation of a methylation profile of the tumor DNA, wherein the profile comprises the methylation status of multiple loci. The methylation profile of the subject sample is then compared to the methylation profile from normal controls generated by the same technique to determine if the methylation profile from the subject sample indicates the presence of a tumor. In an embodiment of the invention, the tumor DNA probe and the normal DNA probe are labeled with two different labels and the hybridization of labeled probes is to one array.

In an embodiment of the invention, the subject DNA sample to be used in the methods of the invention, is derived from plasma or serum. In yet another embodiment of the invention, the methylation specific enzyme is HpyCh4-IV, ClaI, AclI or BstBI. In one embodiment, the methylation specific enzyme is HpyCh4-IV. In another embodiment of the invention, the linker-mediated PCR amplification is performed for about 5 to about 15 cycles. In another embodiment of the invention, the linker-mediated PCR amplification is performed for about 10 cycles. In an embodiment of the invention, exonuclease digestion with Bal-31 is performed following the circularization step.

One embodiment of the invention provides a kit containing the necessary reagents to perform the methods of the present invention along with instructions In one embodiment the kit comprises reagents and instructions for detecting and identifying hypomethylated regions in tumor DNA. In another embodiment, the kit provides reagents and instructions for screening a patient for the presence of tumors by the methods of the present invention. In one embodiment the kit comprises the methylation sensitive enzyme, the linker DNA, the PCR primers for linker-mediated PCR, the restriction enzyme for removing the linkers from the PCR products, the microarray for the detection of tumor related hypomethylated regions and instructions for performing the process.

One embodiment provides a microarray for the detection of hypomethylated regions wherein the microarray comprises oligonucleotides selected by (a) parsing the genome into segments that are bounded by two sites for the methylation sensitive restriction enzyme in question (ACGT for HpyCh4-IV) and less than 500 base pairs long; (b) utilizing an algorithm to analyze the sequence of these fragments, with the goal of finding suitable sequence for representation on the microarray. For example, appropriate oligonucleotides will have one or more of the following characteristics: (i) greater than about 40 nucleotides of unique sequence, or greater than about 60 nucleotides of unique sequence; (ii) a GC of about 40% to about 60%, and (iii) should not contain significant repetitive or simple sequences, for example runs of greater than about 15 of a single base. In one embodiment, the microarray comprises a subset of these oligonucleotides that are useful in the detection of tumor associated hypomethylated DNA. In one embodiment, this subset of oligonucleotides is identified by (i) separately preparing methylation-sensitive representations from tumor and normal DNA using the method described above; (ii) labeling the tumor DNA and control DNA to produce labeled tumor DNA probes and labeled normal DNA probes; (iii) hybridizing the labeled DNA probes to arrays of oligonucleotides, wherein said array of oligonucleotides corresponds to predicted restriction fragments, or portions thereof, for a given methylation-sensitive enzyme; (iv) comparing the relative intensity of the normal and tumor derived probes with each other to identify oligonucleotides that detects the differential amount of tumor DNA probe; (v) identifying the hybridized oligonucleotide from step (iv) as a corresponding to tumor-specific hypomethylated region; and (vi) comparing the identified tumor-specific hypomethylated regions from multiple patients to determine a subset of oligonucleotides that are useful in detecting tumors in patients.

### BRIEF DESCRIPTION OF THE DRAWINGS

FIG. 1. Methylation-microarray comparison of plasma DNA from a subject with ovarian cancer and a normal control. A ˜120 kb region of chromosome 21 containing 112 segments is shown. Positive intensity ratios indicate more relative signal from the cancer sample and negative ratios indicate increased relative signal from the normal sample. The very sharply demarcated cluster of high contrast signals is striking and almost certainly reflects an underlying difference between two samples.

### DETAILED DESCRIPTION OF THE INVENTION

The present invention provides a method of selectively amplifying hypomethylated tumor DNA sequences derived from a subject for detection of cancer. This method utilizes differential methylation to allow for the selective amplification of tumor specific sequences from DNA mixtures that contain a high proportion of normal host DNA. The invention also provides methods of using the amplified tumor DNA sequences for evaluation of methylation.

Differences in Methylation of Tumor and Non-Tumor DNA

As discussed above, the present invention relies on the difference of methylation between tumor and control DNA. Control DNA is understood to be from normal (cancer free) individuals. DNA methylation is an epigenetic event that affects cell function by altering gene expression and refers to the covalent addition of a methyl group, catalyzed by DNA methyltransferase (DNMT), to the 5-carbon of cytosine in a CpG dinucleotide.

The methods of the present invention provide for selective amplification of hypomethylated tumor DNA from a subject derived DNA sample utilizing the methylation differences between tumor DNA and non-tumor DNA. As noted previously, generally the method involves the steps of: isolating DNA from a subject; subjecting the isolated DNA to linker-mediated PCR; circularization of the amplified PCR products; exonuclease digestion; and finally isothermal rolling circle amplification. This method generates methylation-sensitive representations of the tumor DNA, i.e., an amplified reproduction of the tumor DNA based on methylation differences between the tumor DNA and the non-tumor patient DNA.

The methods described herein may be applied to DNA samples derived from cells or cellular materials from a subject. Any method known in the art for collection or isolation of the desired cells or materials can be used. In one embodiment, circulating nucleic acids (CNAs) are derived from the serum or plasma of a subject.

Linker-Mediated PCR

Generally, linker-mediated PCR begins with digesting DNA with a restriction enzyme and ligating double stranded linkers to the digested ends. PCR is then performed with a primer that corresponds to the linker and fragments up to about 1.5 kb are amplified. (See Saunders, Glover et al. 1989; Lisitsyn, Leach et al. 1994). Using this technique, it has been possible to amplify DNA from a single cell and to subsequently detect aneuploidy by using the amplified product to perform comparative hybridization. (Klein, Schmidt-Kittler et al. 1999). In another study, amplified representations were used to detect single genomic copy number variations by using them as hybridization probes to BAC microarrays. (Guillaud-Bataille, Valent et al. 2004).

In this method, the frequency of digestion of the restriction enzyme determines the complexity of the amplified product that results. By choosing an enzyme that cuts infrequently, the complexity of the amplified representation can be reduced to a fraction of the starting genomic DNA making the subsequent hybridization step much easier to perform. This technique has been particularly useful in settings where one wishes to perform comparative hybridizations between two complex genomic sources. A striking example is a technique called “ROMA” (Representational Oligonucleotide Microarray Analysis) that has been instrumental in revealing a high degree of genomic copy number variation in humans. (Lucito, Healy et al. 2003; Sebat, Lakshmi et al. 2004; Jobanputra, Sebat et al. 2005) Lucito, R., et al., Genome Res. 13:2291-305 (2003); Sebat, J., et al., Science 305:525-8 (2004); Jobanputra, V., et al., Genet Med 7:111-8 (2005).

Accordingly, in the linker-mediated PCR step of the present invention, a sample of DNA is obtained and digested with a CpG methylation sensitive enzyme to form digested DNA with digested ends. In one embodiment, the DNA sample is mixed, comprising host and tumor DNA.

By using a CpG methylation sensitive restriction enzyme to cleave DNA prior to linker ligation, amplification of fragments bounded by unmethylated sites is favored. In a setting in which there is a mixture of DNAs from two different sources, one less methylated than the other, digestion with a methylation sensitive enzyme followed by linker ligation and amplification allows the selective amplification of fragments defined by differentially methylated sites. This idea has been used in conjunction with “representational difference analysis” to probe methylation differences between normal and cancerous tissues. (See Ushijima, Morimura et al. 1997; Kaneda, Takai et al. 2003). Methylation sensitive enzymes are known in the art and include, but are not limited to, HpyCh4-IV, ClaI, AclI, and BstBI.

After the DNA obtained from the mixed sample is digested with a methylation specific enzyme as discussed above, the DNA is then ligated to linkers. In one embodiment the linkers have a built in restriction site or part of a restriction site, which will later be used to provide compatible sticky ends necessary for amplification of purified PCR products, for example, a the sticky ends may be used in a circularization step for rolling circle amplification. A restriction enzyme site that produces sticky ends upon digestion is preferred. For example, MluI provides sticky ends.

After ligating the linker, the resulting DNA is amplified using primers that bind to a site within the linker. PCR amplification is then carried out. The number of cycles may vary. In one embodiment, the number of cycles will create a size-selected representation of digested fragments. In one embodiment of the invention, about 5 to about 15 cycles of amplification are carried out. In one embodiment, about 8 to about 14 cycles of amplification are carried out. In a one embodiment, about 10 cycles of amplification are carried out. In one embodiment, one or more of the PCR primers are conjugated with a moiety useful in subsequent purification steps. In one embodiment the moiety is biotin.

Purification of the Linker-Mediated PCR Products

The primers used for linker-mediated PCR may incorporate a moiety useful for purification of the PCR products. In one embodiment, the PCR primer is biotinylated and the PCR products are isolated using a biotin binding protein linked to a support. Biotin binding proteins include e.g. avidin, streptavidin, and NeutrAvidin. In one embodiment the biotin binding protein is streptavidin. In one embodiment, the support is agarose, separose, or magnetic beads. In one embodiment, the PCR primers for linker-mediated PCR are biotinylated and the resulting PCR products are purified using streptavidin linked to magnetic beads. Other components that are not bound to the support can then be washed away. The amplified PCR product is then freed from the support using a restriction endonuclease that recognizes a restriction site contained partially or entirely within the linkers. In one embodiment the restriction enzyme is MluI.

The recognition sequence for MluI (ACGCGT) overlaps with the recognition sequence for the methylation sensitive restriction enzyme HpyCh4-IV (ACGT) such that when the DNA is cleaved with HpyCh4-IV, and subsequently ligated to a linker that includes the sequence, CGCGT, at the 5′ end, the restriction site for MluI is created. Following linker-mediated PCR and binding of the PCR products to a support via a moiety such as biotin, when MluI is used to free the PCR products from the linkers and support, non-specific amplification products will be largely remain bound to the linker and support because they do not contain the entire MluI recognition sequence. Thus, the specific linker-mediated PCR products can be purified from the non-specific amplification products, which remain bound to the support.

In another embodiment of the present invention, After the cycles of amplification are carried out, the amplified products are then digested with an enzyme that cleaves off the linker. For example, if the linker introduces a MluI site, then the products would be subjected to a MluI enzyme digest. Following digestion to cleave the linker, low molecular weight DNA (linker and primer DNA) is removed. Any suitable method to remove low molecular weight DNA may be used, such as agarose gel purification or column purification. Again, the use of the combination of HpyCh4-IV and MluI along with the appropriate linker sequence as described above ensures that non-specific amplification products are not freed from the linkers, and therefore not available for subsequent amplification steps.

Amplification of the Purified PCR Products

Once the linker-mediated PCR products are cleaved and purified from the linkers, the purified DNA is then diluted. This DNA is then treated with T4 DNA ligase overnight to allow circularization by allowing ligation of the sticky ends created by the earlier enzyme digest. By digesting and ligating in a very dilute solution (e.g., 0.5 ml in 1× ligation buffer), intra-molecular self-ligation (circularization) of molecules with compatible sticky ends is strongly favored. The original starting DNA that has been melted and partially re-annealed multiple times (during the PCR amplification) is very inefficiently digested and circularized. Further, the non-specifically amplified products that lack appropriate ends will also be highly unlikely to form covalently closed circles.

The ligations are then used as template for isothermal rolling circle amplification. Isothermal rolling circle amplification is known in the art and is generally a one cycle amplification of circular DNA using exonuclease-resistant random primers and a DNA polymerase with great processivity. Any isothermal rolling circle amplification procedure may be used. A commonly known kit is available from Amersham and is used following the manufacturer's recommendations. The rolling circle amplification results in formation of concatenated structures consisting of multiple copies of the circular template.

In one embodiment, after freeing the purified PCR products from the linker, the products are further amplified using an additional ligation mediated PCR step.

Exonuclease Digestion

After the circularized DNA is precipitated (using methods commonly known in the art) and resuspended in a suitable buffer such as water, the ligation mixture can be treated to remove non-specific PCR products by extensive digestion with an exonuclease that attacks the ends of single stranded and double stranded DNA (e.g. nuclease Bal-31). The circular molecules created by ligation are resistant to digestion, but extensive digestion will reduce any linear molecules to single nucleotides. This digestion is used to thus eliminate the starting genomic DNA as well as non-specifically amplified products. Alternatively, instead of a single exonuclease such as Bal-31, a mixture of exonucleases could be used. For example, one enzyme attacks single stranded DNA (mung bean exonuclease) and the other enzyme attacks double stranded DNA (Lambda exonuclease) and wherein neither of the enzymes have endonuclease activity and neither cleaves double stranded DNA at nicks. By the term extensive digestion, it is meant that a sufficient amount of enzyme is used so as not to be limiting and that the time allowed for digestion is long enough not to be limiting. For example, in one embodiment 2 units of Bal-31 nuclease is used in the digestion mixture and allowed to proceed for 45 minutes. The units are defined functionally as the amount of enzyme needed to digest 400 bases of linear DNA in a 40 ng/μl solution in 10 minutes.

Array Design

The present invention further provides for the use of oligonucleotide microarrays for identification of tumor-specific hypomethylated regions of the genome. In a specific embodiment, the method comprises, (i) separately preparing methylation-sensitive representations from cell-free plasma DNA from subjects and normal controls using the method described above; (ii) labeling the tumor DNA and control DNA to produce labeled tumor DNA probes and labeled normal DNA probes; (iii) hybridizing the labeled DNA probes to arrays of oligonucleotides, wherein said array of oligonucleotides corresponds to predicted restriction fragments, or portions thereof, for a given methylation-sensitive enzyme; (iv) comparing the relative intensity of the normal and tumor derived probes with each other to identify oligonucleotides that detects the differential amount of tumor DNA probe; (v) identifying the hybridized oligonucleotide from step (iv) as a corresponding to tumor-specific hypomethylated region.

The present invention further provides a method for detecting cancer in a subject through the use of microarrays. The method comprises selective amplification of DNA derived from a subject sample and a normal control using the method described above followed by labeling the amplified DNA to produce labeled DNA probes wherein the subject derived probes and normal control derived probes have different labels (e.g., different fluorochromes). The labeled DNA probes are hybridized to an oligonucleotide array, wherein said array of oligonucleotides correspond to predicted restriction fragments for the methylation specific enzyme. The array data is analyzed to ascertain the relative signal strengths from the hybridized probes and determine which segments are preferentially amplified from cancer subjects vs. normal controls. Such analysis will lead to the generation of a methylation profile of the tumor DNA, wherein the profile comprises the methylation status of multiple loci. The methylation profile of the subject sample is then compared to the methylation profile from normal controls generated by the same technique to determine if the methylation profile from the subject sample indicates the presence of a tumor. In one embodiment the subject and control probes are hybridized to two separate arrays.

The arrays to be used in the practice of the invention may be generated using methods well known to those of skill in the art. In one embodiment of the invention, the arrays will contain nucleic acid fragments generated through enzymatic digestion of genomic DNA with the methylation sensitive enzyme utilized in the selective amplification step. In another embodiment, the oligonucleotides on the array correspond to all or a subset of the nucleic acid fragments, or a portion thereof, that could be generated by the methylation sensitive restriction enzyme (i.e., the fragments that could be generated if the DNA was entirely unmethylated). In one embodiment, the oligonucleotides on the microarray may be fabricated in any manner known in the art for example synthesized in situ (on the microarray slide) or spotted on the microarray slide.

Early studies have shown that methylation differences are strikingly more common in gene-rich portions of the genome. Therefore, in order to maximize the likelihood that methylation differences will be found, an array design can be used in the practice of the invention that targets areas in the genome that have high gene content.

For example, in a non-limiting embodiment of the invention, each chromosome may be divided into bins of 10⁶ bp, starting from one telomere and extending to the other. The percentage of total sequence occupied by exons of known or predicted genes in each of these 3000 sequence bins will then be determined using information from the UCSC browser, and all bins will be ranked according to this statistic. Those bins with the highest exon content will then be selected for representation in the array. Gene-rich segments of 10⁶ by each contain about 2000 HpyCh4-IV fragments that meet size criteria for inclusion in the array, and since about 120,000 such fragments can be represented in a standard 385K array, the array will represent about 60 such sequence bins, or about 60×10⁶ bases, corresponding to about 2% of genomic DNA.

In order to provide robust hybridization data, each genomic HpyCh4-IV fragment can be represented on the array by different oligonucleotides that hybridize with these fragments. If possible, it is usually beneficial to include about 3 different oligonucleotides onto the array for each genomic fragment. Commercially available services are available to screen the entire human genome sequence for all possible “longmer” oligonucleotides that meet a series of criteria for inclusion in genomic microarrays. Suitable segments are unique, free of runs of simple sequence, and have an appropriate predicted melting temperature. A commercial service can be provided with the coordinates of the 200,000 fragments as defined above, and they will determine which of the fragments contain at least 3 of their previously established suitable oligonucleotides. Since current arrays have space for 385K oligonucleotides (e.g., NimbleGen arrays), and since each HypCh4-IV fragment will be represented by 3 oligonucleotides, one array is sufficient to represent about 125K fragments. For the purpose of determining background hybridization, a series of 4000 random sequence oligonucleotides are included in each array.

Array Hybridization

Array hybridizations may be carried out by commercial services according to their standard protocols. In one embodiment of the invention, hybridizations are performed as two color “comparisons”, with the “test” DNA labeled with one fluorochrome and the “control” DNA labeled with a second fluorochrome. This approach minimizes artifacts and uniformity problems since the exact same experimental conditions apply to both the “test” and “control” samples. As discussed above, the control for each hybridization will be a different normal subject. It should be understood that, because the data are generated by comparative hybridization, data analysis is not restricted by this aspect of the experimental design. Normalized intensities associated with each array address can be compared across all hybridizations, making it possible, for example, to establish a set of array addresses that are unlikely to result in an above threshold signal in any normal individual.

The microarray detection may be performed by any method known in the art. The DNA samples (i.e., the methylation-sensitive representations) may be labeled with labels useful for detection on a microarray including, but not limited to, fluorescent labels, luminescent labels, gold particle labels, and electrochemical labels.

Data Analysis

Comparative hybridization to microarrays has been used extensively to profile gene expression as well as to identify genomic copy number variation, and there are abundant methods of data analysis for microarray data of this type. In the present invention, the data may be used to assess genomic distribution of cancer-specific differential methylation and to assess overall differences in relative signal intensity between microarray data sets.

Existing bioinformatics methods for evaluating alterations in genome copy number, for example, may be used for data analysis, included “thresholding” (Vissers, de Vries et al. 2003), hidden Markov models (Sebat, Lakshmi et al. 2004), hierarchical clustering using genomic position (Wang, Kim et al. 2005) and, most recently, a technique known as maximum-a-posteriori or “MAP” (Daruwala, Rudra et al. 2004). Although these methods have been developed for the detection of copy number variations rather than methylation differences, the general problems are similar, and the methods are readily adaptable to the type of data that our arrays will generate.

Once individual data sets have been analyzed for the presence of reliable clusters of differential signal, comparisons between data sets aimed at discriminating cancer from normal can be performed. Several published studies that have specifically addressed this type of comparison in the context of microarray/methylation data. For instance, in a study involving a small-scale microarray assay that consists of 8000 CpG island loci immobilized glass slides, hierarchical clustering was able to identify two different groups of ovarian tumor, and this correlated to clinical parameters (Wei, Chen et al. 2002). In a subsequent publication (Wei, Balch et al. 2006), the same group reported expanded this analysis by using Significance Analysis of Microarrays (SAM) (Tusher, Tibshirani et al. 2001) and Prediction Analysis of Microarray (PAM) (Tibshirani, Hastie et al. 2002) as well as other bioinformatics techniques for interpreting microarray data on tumor methylation. In general, there are a large variety of methods for assessing similarities between different microarray data sets that are well known to those of skill in the art.

All references referred to herein are incorporated in their entirety.

### EXAMPLES

### Example 1

### Identification of a Tumor Associated Hypomethylated Region

To test whether methylation profiles of CNA from subjects with ovarian tumors is different from that of normal controls, frozen serum samples were obtained from women who had their blood drawn prior to exploratory surgery for suspected ovarian cancer. Similar samples were obtained from women without cancer. DNA was prepared from 1 ml of cell-free serum by a standard method and the entire resulting sample was subjected to methylation-sensitive amplification as described above. One such pair of samples was submitted to NimbleGen for hybridization to an array that had previously been used for analysis of trophoblast methylation.

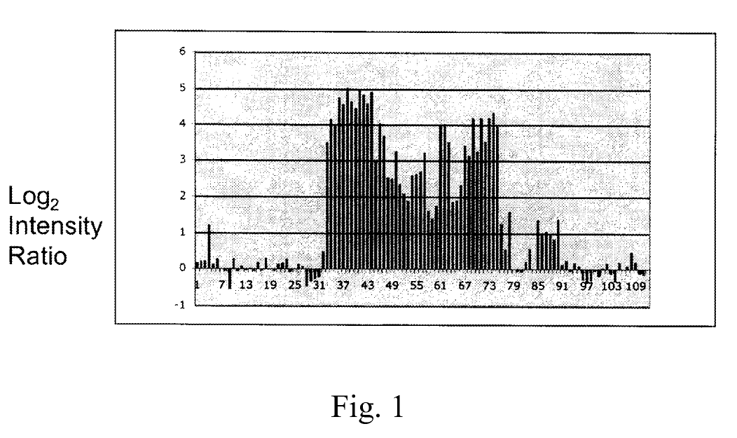

The data indicated that both amplifications (cancer and normal) resulted in measurable signal (defined as >3sd above background) from ˜5% of array addresses. Additionally, in ˜70% of these cases, the log₂-ratio of the signals is less than |1.5|, indicating that even though that segment amplified from both cancer as well as normal, there is little or no differential amplification. This data demonstrates success in both amplifying serum DNA and in using amplified representations to obtain signal from a microarray. It should be noted that non-specific amplification would be expected to result in scattered or randomly placed hybridization signals, which is not observed. Furthermore, regions of differential amplification clearly occur in clusters. FIG. 1 shows the data from a small region on chromosome 21 that contains a cluster of high contrast signals from the cancer specimen. Note that at least 40 adjacent segments are differentially amplified and that log₂-ratios are as high as 5, indicating 32 fold differential amplification. This is extremely unlikely to be due to experimental artifact and therefore most likely represents detection of true methylation differences between the original samples. This is one of approximately 50 clusters (>3 adjacent segments) with log 2 ratio of signal intensity >2.

The experiments described in the following Examples are intended to represent possible embodiments of the present invention. It is understood that the materials and amounts do not limit the scope of the invention.

### Example 2

### Development of a Comparison Panel

In order to facilitate detection and diagnosis using the methods of the present invention, normal and specific cancer patient populations can be compared to develop a methylation profile associated with a particular type of cancer. The methods of the present invention can be used to create such a methylation profile.

DNA is isolated from the serum or plasma of known cancer patients and normal controls using standard methods (Johnson, K. L., et al., Clin. Chem. 50:516-21 (2004)). Briefly, 10 ml of patient blood is centrifuged two times to remove cells. The resulting plasma is passed over a DNA binding membrane. The DNA is removed from the membrane and the resulting DNA is digested with HpyCh4-IV.

DNA linkers are annealed and ligated to the digested DNA. The linkers are designed to create a MluI restriction site when ligated to DNA digested with HpyCh4-IV. The linker-mediated PCR is performed as described by Guillaud-Bataille, M., et al. Nucleic Acids Res. 32e112 (2004)) with 10 cycles of PCR, utilizing biotinylated primers.

Following the PCR, the products purified utilizing streptavidin coated magnetic beads. After the PCR products are bound to the beads and washed, they are digested with MluI to remove the linker sequences (and beads) from the amplified DNA. The amplified DNA is circularized by diluting the DNA to promote intramolecular ligation and treating with T4 DNA ligase.

The ligation products are then used as a template for isothermal rolling circle amplification using a commercial kit (e.g., Amersham) and following the manufacturer's instructions.

DNA prepared by the above method may then be labeled and hybridized to a custom made oligonucleotide microarray. Each oligonucleotide on the array corresponds to one of the DNA restriction fragments that could be theoretically created during the first digestion step using the methylation sensitive enzyme. Hybridizations are performed as two color “comparisons”, with the “test” DNA labeled with one fluorochrome (e.g., Cy3) and the “control” DNA labeled with a second fluorochrome (e.g., Cy5). The control for each hybridization will be a different normal subject.

The intensity of signal at each array address is dependent on the amount of probe that corresponds to the address. Thus, array addresses for which signal intensity is high are relatively less methylated. Through a comparison of microarray data from normal controls to those with tumors, a typical methylation profile of the tumor type is derived empirically. Differences in methylation identified by comparing known cancer subjects to non-cancer will be used to develop criteria, which will be validated by applying them prospectively. This method can be used to develop a methylation profile for a variety of tumors including, but not limited to ovarian, lung, prostate, and breast.

### Example 3

### Making a Microarray for Detection of Hypomethylated, Tumor-Associated DNA

The genome is parsed into segments that are bounded by two sites for the methylation sensitive restriction enzyme in question (ACGT for HpyCh4-IV) and less than 500 base pairs long. This provides a list of DNA segments that might be amplified from a serum or plasma DNA sample. An algorithm is used to analyze the sequence of these fragments, with the goal of finding suitable sequence for representation on the microarray. For example, appropriate oligonucleotides will have one or more of the following characteristics: (i) greater than about 40 nucleotides of unique sequence, or greater than about 60 nucleotides of unique sequence; (ii) a GC of about 40% to about 60%, and (iii) should not contain significant repetitive or simple sequences, for example runs of greater than about 15 of a single base. The array contains oligonucleotides chosen in this way with each oligonucleotide on the array representing one genomic segment that could have been amplified by the method of the present invention. Such an array is useful for the detection of tumor associated hypomethylated regions, the development of methylation profile for tumors, and for the screening for tumors using the methods of the present invention.

Once the above microarray has been used to identify tumor associated regions of DNA that are hypomethylated, either in tumors in general or in one or more specific tumor types, microarrays comprising oligonucleotides designed to detect just those DNA regions that are typically associated with tumors in general or with one or more types of tumors may be generated for detection of tumor associated methylation differences at those loci using the methods in Example 4.

### Example 4

### Method of Diagnosing Cancer using the Present Invention

DNA is isolated from patient serum or plasma using standard methods (Johnson, K. L., et al., Clin. Chem. 50:516-21 (2004)). Briefly, 10 ml of patient blood is centrifuged two times to remove cells. The resulting plasma is passed over a DNA binding membrane. The DNA is removed from the membrane and the resulting DNA is digested with HpyCh4-IV.

DNA linkers are annealed and ligated to the digested DNA. The linkers are designed to create a MluI restriction site when ligated to DNA digested with HpyCh4-IV. The linker-mediated PCR is performed as described by Guillaud-Bataille, M., et al. Nucleic Acids Res. 32e112 (2004)) with 10 cycles of PCR, and biotinylated primers.

Following the PCR, the products purified utilizing streptavidin coated magnetic beads. After the PCR products are bound to the beads and washed, they are digested with MluI to remove the linker sequences (and beads) from the amplified DNA. The amplified DNA is circularized by diluting the DNA to promote intramolecular ligation and treating with T4 DNA ligase.

After ligation, the are then used as a template for isothermal rolling circle amplification using a commercial kit (e.g., Amersham) and following the manufacturer's instructions.

DNA prepared by the above method is labeled and hybridized to a custom made oligonucleotide microarray. Hybridizations are performed as two color “comparisons”, with the patient DNA labeled with one fluorochrome and the control DNA labeled with a second fluorochrome. Each oligonucleotide on the array corresponds to one of the DNA restriction fragments that could be theoretically created during the first digestion step using the methylation sensitive enzyme. The intensity of signal at each array address is dependent on the amount of probe that corresponds to the address. Thus, array addresses for which signal intensity is high are relatively less methylated. Methylation/microarray results from samples obtained from subjects where cancer status is unknown is compared with the body of normal and cancer data derived in Example 2. Deviations from normal are indicative of cancer. Methods for comparing microarray data are known in the art.

Following a positive result, the patient can be screened by an appropriate screen to confirm the cancer diagnosis, for example an MRI.

These methods are applicable to the detection of a variety of tumor types, including but not limited to ovarian, lung, prostate, and breast. In addition, this method may be used as a general screening test.

### REFERENCES

Chen, X., H. Bonnefoi, et al. (1999). “Detecting tumor-related alterations in plasma or serum DNA of patients diagnosed with breast cancer.” Clin Cancer Res 5(9): 2297-303.
Daruwala, R. S., A. Rudra, et al. (2004). “A versatile statistical analysis algorithm to detect genome copy number variation.” Proc Natl Acad Sci USA 101(46): 16292-7.
Fleischhacker, M. and B. Schmidt (2007). “Circulating nucleic acids (CNAs) and cancer—A survey.” Biochim Biophys Acta 1775(1): 181-232.
Guillaud-Bataille, M., A. Valent, et al. (2004). “Detecting single DNA copy number variations in complex genomes using one nanogram of starting DNA and BAC-array CGH.” Nucleic Acids Res 32(13): e112.
Herman, J. G., J. R. Graff, et al. (1996). “Methylation-specific PCR: a novel PCR assay for methylation status of CpG islands.” Proc Natl Acad Sci USA 93(18): 9821-6.
Jobanputra, V., J. Sebat, et al. (2005). “Application of ROMA (representational oligonucleotide microarray analysis) to patients with cytogenetic rearrangements.” Genet Med 7(2): 111-8.
Kaneda, A., D. Takai, et al. (2003). “Methylation-sensitive representational difference analysis and its application to cancer research.” Ann N Y Acad Sci 983: 131-41.
Klein, C. A., O. Schmidt-Kittler, et al. (1999). “Comparative genomic hybridization, loss of heterozygosity, and DNA sequence analysis of single cells.” Proc Natl Acad Sci USA 96(8): 4494-9.
Kopreski, M. S., F. A. Benko, et al. (2000). “Somatic mutation screening: identification of individuals harboring K-ras mutations with the use of plasma DNA.” J Natl Cancer Inst 92(11): 918-23.
Laird, P. W. (2005). “Cancer epigenetics.” Hum Mol Genet 14 Spec No 1: R65-76.
Lisitsyn, N. A., F. S. Leach, et al. (1994). “Detection of genetic loss in tumors by representational difference analysis.” Cold Spring Harb Symp Quant Biol 59: 585-7.
Lucito, R., J. Healy, et al. (2003). “Representational oligonucleotide microarray analysis: a high-resolution method to detect genome copy number variation.” Genome Res 13(10): 2291-305.
Saunders, R. D., D. M. Glover, et al. (1989). “PCR amplification of DNA microdissected from a single polytene chromosome band: a comparison with conventional microcloning.” Nucleic Acids Res 17(22): 9027-37.
Sebat, J., B. Lakshmi, et al. (2004). “Large-scale copy number polymorphism in the human genome.” Science 305(5683): 525-8.
Sozzi, G., D. Conte, et al. (2001). “Analysis of circulating tumor DNA in plasma at diagnosis and during follow-up of lung cancer patients.” Cancer Res 61(12): 4675-8.
Sozzi, G., K. Musso, et al. (1999). “Detection of microsatellite alterations in plasma DNA of non-small cell lung cancer patients: a prospect for early diagnosis.” Clin Cancer Res 5(10): 2689-92.
Tibshirani, R., T. Hastie, et al. (2002). “Diagnosis of multiple cancer types by shrunken centroids of gene expression.” Proc Natl Acad Sci USA 99(10): 6567-72.
Trombino, S., M. Neri, et al. (2005). “Mutations in K-ras codon 12 detected in plasma DNA are not an indicator of disease in patients with non-small cell lung cancer.” Clin Chem 51(7): 1313-4.
Tusher, V. G., R. Tibshirani, et al. (2001). “Significance analysis of microarrays applied to the ionizing radiation response.” Proc Natl Acad Sci USA 98(9): 5116-21.
Ushijima, T., K. Morimura, et al. (1997). “Establishment of methylation-sensitive-representational difference analysis and isolation of hypo- and hypermethylated genomic fragments in mouse liver tumors.” Proc Natl Acad Sci USA 94(6): 2284-9.
Vissers, L. E., B. B. de Vries, et al. (2003). “Array-based comparative genomic hybridization for the genomewide detection of submicroscopic chromosomal abnormalities.” Am J Hum Genet 73(6): 1261-70.
Wang, P., Y. Kim, et al. (2005). “A method for calling gains and losses in array CGH data.” Biostatistics 6(1): 45-58.
Wei, S. H., C. Balch, et al. (2006). “Prognostic DNA methylation biomarkers in ovarian cancer.” Clin Cancer Res 12(9): 2788-94.
Wei, S. H., C. M. Chen, et al. (2002). “Methylation microarray analysis of late-stage ovarian carcinomas distinguishes progression-free survival in patients and identifies candidate epigenetic markers.” Clin Cancer Res 8(7): 2246-52.
Yakubovskaya, M. S., V. Spiegelman, et al. (1995). “High frequency of K-ras mutations in normal appearing lung tissues and sputum of patients with lung cancer.” Int J Cancer 63(6): 810-4.

1. A method for making a methylation-sensitive representation of hypomethylated tumor DNA from a patient sample comprising
a) isolating DNA from a patient sample;
b) digesting the DNA with a methylation sensitive enzyme;
c) ligating the digested DNA to a linker;
d) subjecting the ligated DNA to linker-mediated PCR amplification to obtain PCR products;
e) circularizing the PCR products;
f) amplifying the circularized PCR products to produce a methylation-sensitive representation from the patient DNA.

2. The method of claim 1, wherein the patient sample is plasma or serum.

3. The method of claim 1, wherein the methylation specific enzyme is HpyCh4-IV, ClaI, AclI or BstBI.

4. The method of claim 1, wherein the methylation specific enzyme is HpyCh4-IV.

5. The method of claim 1, wherein the linker-mediated PCR amplification is performed for about 5 to about 15 cycles.

6. The method of claim 1, wherein the linker-mediated PCR amplification is performed for about 10 cycles.

7. The method of claim 1, wherein the PCR products of (d) are purified by precipitation.

8. The method of claim 1, wherein the PCR amplification is performed with biotinylated PCR primers, and the PCR products are purified utilizing a biotin binding protein linked to a support.

9. The method of claim 8, wherein the biotin binding protein is streptavidin.

10. The method of claim 8, wherein the support is selected from the group consisting of agarose, sepharose, and magnetic beads.

11. The method of claim 8, wherein the PCR products are cleaved from the support using a restriction enzyme with a recognition site partly or entirely contained in the linker sequence.

12. The method of claim 1, wherein the circularized PCR products are amplified by rolling circle amplification.

13. The method of claim 1, wherein the tumor is selected from the group consisting of ovarian tumor, lung tumor, prostate tumor, or breast tumor.

14. The method of claim 13, wherein the tumor is an ovarian tumor.

15. A method for making a methylation-sensitive representation of hypomethylated tumor DNA from a patient sample comprising
a) isolating DNA from a patient sample;
b) digesting the DNA with HpyCh4-IV;
c) ligating the digested DNA to a linker, such that an MluI recognition site is created by the ligation of the linkers to the digested DNA;
d) subjecting the digested DNA to linker-mediated PCR amplification with biotinylated primers to obtain PCR products;
e) purifying the amplified PCR products using a biotin binding protein linked to a support;
f) digesting the PCR products with MluI;
g) circularizing the digested DNA; and
h) performing rolling circle amplification to produce a methylation-sensitive representation from the patient DNA.

16. The method of claim 15, wherein the tumor is selected from the group consisting of ovarian tumor, lung tumor, prostate tumor, or breast tumor.

17. The method of claim 16, wherein the tumor is an ovarian tumor.

18. A method for identifying tumor-specific hypomethylated DNA regions comprising,
a) separately preparing methylation-sensitive representations from tumor and normal DNA by;
i) digesting the DNA with a methylation sensitive enzyme;
ii) ligating the digested DNA to a linker;
iii) subjecting the ligated DNA to linker-mediated PCR amplification to obtain PCR products;
iv) circularizing the PCR products; and
v) amplifying the circularized PCR products to produce a methylation-sensitive representation from the DNA;
b) labeling the tumor DNA representation and the normal DNA representation to produce labeled tumor DNA probes and labeled normal DNA probes;
c) hybridizing the labeled DNA probes to arrays of oligonucleotides, wherein said arrays of oligonucleotides correspond to predicted restriction fragments, or portions thereof, for a given methylation-sensitive enzyme;
d) comparing the relative intensity of the signals from the normal and tumor derived probes with each other to identify oligonucleotides that detects the differential amount of tumor DNA probe;
e) identifying the hybridized oligonucleotide from step d as corresponding to tumor-specific hypomethylated region.

19. The method of claim 18, wherein the tumor DNA probe and the DNA probe are labeled with two different labels and wherein the hybridization of labeled probes is to one array.

20. The method of claim 18, wherein the DNA sample is from plasma or serum.

21. A method for detecting the presence of a tumor in a subject comprising
a) preparing a methylation-sensitive representation of patient DNA using the method of claim 1;
b) comparing the amount of amplified DNA in the methylation-sensitive representation of step a) with the amount of DNA in the methylation-sensitive representation of normal DNA made by the same method; and
c) identifying an increased amount of amplified DNA in the methylation-sensitive representation of step a) relative to the methylation-sensitive representation from normal DNA as indicative of the presence of a tumor.

22. The method of claim 21, wherein the patient DNA representation and the normal DNA representation are labeled and hybridized to one or more oligonucleotide microarrays.

23. The method of claim 21, wherein the tumor is selected from the group consisting of ovarian tumor, lung tumor, prostate tumor, or breast tumor.

24. The method of claim 23, wherein the tumor is an ovarian tumor.

25. A method of making a microarray for detecting hypomethylated tumor DNA in a sample of mixed tumor DNA and normal DNA comprising:
a) identifying tumor-specific hypomethylated DNA regions according to claim 18;
b) selecting a tumor-specific hypomethylated DNA region and at least one oligonucleotide that hybridizes to the tumor-specific hypomethylated DNA region; and
c) preparing a microarray comprising the selected oligonucleotide.

26. The method of claim 25, wherein the tumor-specific hypomethylated DNA region is selected as being hypomethylated in multiple tumor samples.

27. The method of claim 26, wherein the multiple tumor samples are samples from subjects having tumors of the same type.

28. The method of claim 26, wherein the multiple tumor samples are samples from subjects having different tumor types.

29. The method of claim 25, wherein two or more different oligonucleotides are selected in step (b) that hybridize to the tumor-specific hypomethylated DNA region.

30. The method of claim 25, wherein multiple tumor-specific hypomethylated DNA regions are selected in step (b).

31. The method of claim 25, wherein the microarray further comprises one or more oligonucleotide controls that hybridize to DNA regions that are not hypomethylated in tumor DNA.

32. The method of making a microarray of claim 25, wherein the oligonucleotides are selected to detect loci that are hypomethylated in tumors selected from the group consisting of ovarian tumors, prostate tumors, breast tumors, lung tumors or any combination of these tumor types.

33. The method of making a microarray of claim 25, wherein the oligonucleotides are selected to detect loci that are hypomethylated in ovarian tumors.

34. A microarray made by the method of claim 25.

35. A method of making a microarray for detecting methylation differences between tumor DNA and normal DNA comprising
a) isolating DNA from a patient sample, wherein the patient has been diagnosed as having a tumor;
b) digesting the DNA with a methylation specific enzyme;
c) ligating the digested DNA with a linker;
d) subjecting the digested DNA to linker-mediated PCR amplification to obtain amplified PCR products;
e) removing linker and primer DNA from the amplification products;
f) circularizing the amplified PCR products;
g) subjecting the products from step f to isothermal rolling circle amplification to selectively amplify tumor DNA to produce methylation-sensitive representations from tumor DNA;
h) labeling the tumor DNA to produce labeled tumor DNA probes;
i) hybridizing the labeled DNA probe to an oligonucleotide array, wherein said array of oligonucleotides correspond to predicted restriction fragments for the methylation specific enzyme;
j) generating a methylation profile of the tumor DNA, wherein the profile comprises the methylation status of multiple loci;
k) comparing the methylation profile of multiple normal and patient samples to identify loci that are hypomethylated in tumor DNA; and
l) generating a microarray comprising oligonucleotides designed to detect loci that are hypomethylated in tumor DNA.

36. A method for making a methylation-sensitive representation of hypomethylated tumor DNA from a patient sample comprising
a) isolating DNA from a patient sample;
b) digesting the DNA with a methylation sensitive enzyme;
c) ligating the digested DNA to a linker;
d) subjecting the ligated DNA to linker-mediated PCR amplification to obtain PCR products;
e) treating the PCR products with T4 ligase to provide ligated PCR products; and;
f) isothermally amplifying the ligated PCR products to produce a methylation-sensitive representation from tumor DNA.

37. A method for identifying tumor-specific hypomethylated DNA regions comprising,
a) separately preparing methylation-sensitive representations from tumor and normal DNA by;
i) digesting the DNA with a methylation sensitive enzyme;
ii) ligating the digested DNA to a linker;
iii) subjecting the ligated DNA to linker-mediated PCR amplification to obtain PCR products;
iv) treating the PCR products with T4 ligase to provide ligated PCR products; and
v) isothermally amplifying the ligated PCR products to produce a methylation-sensitive representation from the DNA;
b) labeling the tumor DNA representation and the normal DNA representation to produce labeled tumor DNA probes and labeled normal DNA probes;
c) hybridizing the labeled DNA probes to arrays of oligonucleotides, wherein said arrays of oligonucleotides correspond to predicted restriction fragments, or portions thereof, for a given methylation-sensitive enzyme;
d) comparing the relative intensity of the signals from the normal and tumor derived probes with each other to identify oligonucleotides that detects the differential amount of tumor DNA probe; and
e) identifying the hybridized oligonucleotide from step d as corresponding to tumor-specific hypomethylated region.

38. A method for detecting the presence of a tumor in a subject comprising
a) preparing a methylation-sensitive representation of patient DNA using the method of claim 26;
b) comparing the amount of amplified DNA in the methylation-sensitive representation of step a) with the amount of amplified DNA in a methylation-sensitive representation of normal DNA made by the same method; and
c) identifying an increased amount of amplified DNA in the methylation-sensitive representation of step a) relative to the methylation-sensitive representation of normal DNA as indicative of the presence of a tumor.
